(19) United States
(12) Patent Application Publication
(10) Pub. No.: US 2004/0039198 A1
(43) Pub. Date: Feb. 26, 2004

(54) Compounds

(76) Inventors: Paul E. Bender, Cherry Hill, NJ (US); Joelle L. Burgess, Collegeville, PA (US); James F. Callahan, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/416,761

(22) PCT Filed: Nov. 14, 2001
(86) PCT No.: PCT/US01/43994

(30) Foreign Application Priority Data
Nov. 16, 2000 (US) 60249199

(51) Int. Cl. C07D 417/14; C07D 413/14; C 07D 4 3/14

(52) U.S. Cl. 544/49; 544/105; 544/284; 544/353; 546/272.7

(57) ABSTRACT

This invention relates to 2-pyridyl substituted diarylimidazoles which are inhibitors of the transforming growth factor, (“TGF”)-β signaling pathway, in particular, the phosphorylation of smad2 or smad3 by the type I or activin-like kinase (“ALK”)-5 receptor, methods for their preparation and their use in medicine.

This invention relates to 2-pyridyl substituted diarylimidazoles which are inhibitors of the transforming growth factor, (“TGF”)-β signaling pathway, in particular, the phosphorylation of smad2 or smad3 by the type I or activin-like kinase (“ALK”)-5 receptor, methods for their preparation and their use in medicine.

TGF-β1 is the prototypic member of a family of cytokines including the TGF-βs, activins, inhibins, bone morphogenetic proteins and Müllerian-inhibiting substance, that signal through a family of single transmembrane serine/threonine kinase receptors. These receptors can be divided in two classes, the type I or activin like kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β signaling requires both the ALK and type II receptors. Specifically, the type II receptor phosphorylates the GS domain of the type I receptor for TGF-β, ALK5, in the presence of TGF-β. The ALK5, in turn, phosphorylates the cytoplasmic proteins smad2 and smad3 at two carboxy terminal serines. The phosphorylated smad proteins translocate into the nucleus and activate genes that contribute to the production of extracellular matrix. Therefore, preferred compounds of this invention are selective in that they inhibit the type I receptor and thus matrix production.

Activation of the TGF-β1 axis and expansion of extracellular matrix are early and persistent contributors to the development and progression of chronic renal disease and vascular disease. Border W. A., et al, N. Engl. J. Med., 1994; 331(19), 1286-92. Further, TGF-β1 plays a role in the formation of fibronectin and plasrninogen activator inhibitor-1, components of sclerotic deposits, through the action of smad3 phosphorylation by the TGF-β1 receptor ALK5. Zhang Y., et al, Nature, 1998; 394(6696), 909-13; Usui T., et al, Invest. OphthalmoL Vis. Sci., 1998; 39(11), 1981-9.

Progressive fibrosis in the kidney and cardiovascular system is a major cause of suffering and death and an important contributor to the cost of health care. TGF-β1 has been implicated in many renal fibrotic disorders. Border W. A., et al, N. Engl. J. Med., 1994; 331(19), 128692. TGF-β1 is elevated in acute and chronic glomerulonephritis Yoshioka K., et al, Lab. Invest., 1993; 68(2), 154-63, diabetic nephropathy Yamamoto, T., et al, 1993, PNAS 90, 1814-1818., allograft rejection, HIV nephropathy and angiotensin-induced nephropathy Border W. A., et al, N. Engl. J. Med., 1994; 331(19), 1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular matrix protein and inhibit protease activity by exogenous TGF-β1 in vitro. Second, neutralizing anti-bodies against TGF-β1 can prevent the accumulation of extracellular matrix in nephritic rats. Third, TGF-β1 transgenic mice or in vivo transfection of the TGF-β1 gene into normal rat kidneys resulted in the rapid development of glomerulosclerosis. Kopp J. B., et al, Lab. Invest., 1996; 74(6), 991-1003. Thus, inhibition of TGEF-β1 activity is indicated as a therapeutic intervention in chronic renal disease.

TGF-β1 and its receptors are increased in injured blood vessels and are indicated in neointima formation following balloon angioplasty Saltis J., et al, Clin. Exp. Pharmacol. Physiol., 1996; 23(3), 193-200. In addition TGF-β1 is a potent stimulator of smooth muscle cell (“SMC”) migration in vitro and migration of SMC in the arterial wall is a contributing factor in the pathogenesis of atherosclerosis and restenosis. Moreover, in multivariate analysis of the endothelial cell products against total cholesterol, TGF-β1 receptor ALK5 correlated with total cholesterol (P<0.001) Blann A. D., et at, Atherosclerosis, 1996; 120(1-2), 221-6.

Furthermore, SMC derived from human atherosclerotic lesions have an increased ALK5/TGF-β type II receptor ratio. Because TGF-β1 is over-expressed in fibroproliferative vascular lesions, receptor-variant cells would be allowed to grow in a slow, but uncontrolled fashion, while overproducing extracellular matrix components McCaffrey T. A., et al, Jr., J. Clin. Invest., 1995; 96(6), 2667-75. TGF-β1 was immunolocalized to non-foamy macrophages in atherosclerotic lesions where active matrix synthesis occurs, suggesting that non-foamy macrophages may participate in modulating matrix gene expression in atherosclerotic remodeling via a TGF-p-dependent mechanism. Therefore, inhibiting the action of TGF-β1 on ALK5 s also indicated in atherosclerosis and restenosis.

TGF-β is also indicated in wound repair. Neutralizing antibodies to TGF-β1 have been used in a number of models to illustrate that inhibition of TGF-β1 signaling is beneficial in restoring function after injury by limiting excessive scar formation during the healing process. For example, neutralizing antibodies to TGF-β1 and TGF-β2 reduced scar formation and improved the cytoarchitecture of the neodermis by reducing the number of monocytes and macrophages as well as decreasing dermal fibronectin and collagen deposition in rats Shah M., J. Cell. Sci., 1995, 108, 985-1002. Moreover, TGF-β antibodies also improve healing of corneal wounds in rabbits Moller-Pedersen T., Curr. Eye Res., 1998, 17, 736-747, and accelerate wound healing of gastric ulcers in the rat, Ernst H., Gut, 1996, 39, 172-175. These data strongly suggest that limiting the activity of TGF-β would be beneficial in many tissues and suggest that any disease with chronic elevation of TGF-β would benefit by inhibiting smad2 and smad3 signaling pathways.

TGF-β is also implicated in peritoneal adhesions Saed G. M., et al, Wound Repair Regenteration, 1999 Nov-Dec, 7(6), 504-510. Therefore, inhibitors of ALK5 would be beneficial in preventing peritoneal and sub-dermal fibrotic adhesions following surgical procedures.

Surprisingly, it has now been discovered that a class of 2-pyridyl substituted diarylimidazole compounds function as potent and selective non-peptide inhibitors of ALK5 kinase and therefore, have utility in the treatment and prevention of various disease states mediated by ALK5 kinase mechanisms, such as chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis, liver fibrosis and renal fibrosis, and restenosis.

According to the invention there is provided a compound of formula (I), or a pharmaceutically acceptable salt thereof:

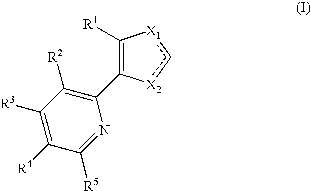

wherein R¹ is phenyl or naphthyl optionally substituted with one or more substituents selected from halo, C₁₋₆alkoxy, C₁₋₆alylthio, C₁₋₆alkyl, Cₗ₋₆haloalkyl, —O—(CH₂)ₘ—Ph, —S—(CH₂)ₘ—Ph, cyano, phenyl, and CO₂R, wherein R is hydrogen or C₁₋₆alkyl and m is 0-3; or phenyl fused with a 5- to 7-membered aromatic or non-aromatic ring wherein said ring contains up to three heteroatorns, independently selected from N, O and S;

R², R³, R⁴ and R⁵ independently represent hydrogen, C₁₋₆alkyl, C₁₋₆alkoxy, C₁₋₆haloalkyl, halo, NH₂, NH—C₁₋₆alkyl or NH(CH₂)ₙ—Ph wherein n is 0-3; or an adjacent pair of R², R³, R⁴ and R⁵ form a fused 6-membered aromatic ring optionally containing up to 2 nitrogen atoms, said ring being optionally substituted by one or more substituents independently selected from C₁₋₆allyl, C₁₋₆alkoxy, C₁₋₆haloalkyl, halo, NH₂, NH—C₁₋₆alkyl or NH(CH₂)ₙ—Ph wherein n is 0-3, and the remainder of R², R³, R⁴ and R⁵ represent hydrogen, C₁₋₆alkyl, C₁₋₆alkoxy, C₁₋₆haloalkyl, halo, NH₂, NH—C₁₋₆alkyl or NH(CH₂)ₙ—Ph wherein n is 0-3; and

one of X₁ and X₂ is N and the other is NR⁶, wherein R⁶ is hydrogen or C₁₋₆alkyl.

As used herein, the double bond indicated by the dotted lines of formula (I), represent he possible tautomeric ring forms of the compounds falling within the scope of this invention. It will be understood that the double bond is to the unsubstituted N atom.

R¹ is preferably phenyl optionally substituted by halo, or R¹ is phenyl fused with a 5- to 7-membered aromatic or non-aromatic ring wherein said ring contains up to three heteroatoms, independently selected from N, O and S, for example R¹ may represent benzo[1,3]dioxolyl, 2,3-dihydrobenzo[1,4]dioxinyl, benzoxazolyl, benzothiazolyl, benzo[1,2,5]oxadiazolyl, benzo[1,2,5]thiadiazolyl or dihydrobenzofuranyl.

Preferably one of R², R³, R⁴ and R⁵ is other than hydrogen, e.g. methyl or halo. More preferably R⁵ is methyl or halo.

When an adjacent pair of R², R³, R⁴ and R⁵ form a fused 6-membered aromatic ring optionally containing up to 2 nitrogen atoms, R⁴ and R⁵ preferably form a fused phenyl ring.

R⁶ is preferably hydrogen.

The compounds of formula (I) preferably have a molecular weight of less than 800.

Particular compounds according to the invention include those mentioned in the examples and their pharmaceutically acceptable salts.

Suitable pharmaceutically acceptable salts of the compounds of formula (I) include, but are not limited to, salts with inorganic acids such as hydrochloride, sulfate, phosphate, diphosphate, hydrobromide, and nitrate, or salts with an organic acid such as malate, maleate, fumarate, tartrate, succinate, citrate, acetate, lactate, methanesulfonate, p-toluenesulfonate, palmitate, salicylate and stearate.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Certain of the compounds of formula (I) may exist in the form of optical isomers, e.g. diastereoisomers and mixtures of isomers in all ratios, e.g. racemnic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis).

Impure preparations of the compounds may be used for preparing the more pure forms used in the pharmaceutical compositions; these less pure preparations of the compounds should contain at least 1%, more suitably at least 5% and preferably at least 10% of a compound of the formula (I) or pharmaceutically acceptable derivative thereof.

The term “C₁₋₆alkyl” as used herein whether on its own or as part of a larger group e.g. C₁₋₆alkoxy, means a straight or branched chain radical of 1 to 6 carbon atoms, unless the chain length is limited thereto, including, but not limited to methyl, ethyl, n-propyl, isopropyl, n-butyl, sec-butyl, isobutyl and tert-butyl.

C₁₋₆haloalkyl groups may contain one or more halo atoms, a particular C₁₋₆haloalkyl group that may be mentioned in CF₃.

The terms “halo” or “halogen” are used interchangeably herein to mean radicals derived from the elements chlorine, fluorine, iodine and bromine.

The term “cycloalkyl” as used herein means cyclic radicals, preferably of 3 to 7 carbons, including but not limited to cyclopropyl, cyclopentyl and cyclohexyl.

The term “ALK5 inhibitor” as used herein means a compound, other than inhibitory smads, e.g. smad6 and smad7, which selectively inhibits the ALK5 receptor preferentially over p38 or type II receptors.

The term “ALK5 mediated disease state” as used herein means any disease state which is mediated (or modulated) by ALK5, for example a disease which is modulated by the inhibition of the phosphorylation of smad 2/3 in the TGF-β1 signaling pathway.

The term “ulcers” as used herein includes but is not limited to, diabetic ulcers, chronic ulcers, gastric ulcers, and duodenal ulcers.

The compounds of formula (I) can be prepared by art-recognized procedures from known or commercially available starting materials. If the starting materials are unavailable from a commercial source, their synthesis is described herein, or they can be prepared by procedures known in the art.

Compounds in which R⁶ is hydrogen may be produced by condensation of an α-aryl(R¹)-tosmic reagent with a 2-pyridyl aldehyde and ammonia to give a diaryl-imidazole (Scheme 1).

Non-selective alkylation of the imidazole nitrogen (using one of the procedures outlined in N. J. Liverton et al; J. Med. Chem., 1999, 42, 2180-2190) with a compound of formula X-R⁶ wherein X is a leaving group, e.g. halo, sulfonate or triflate, will yield both isomers of the compounds of formula (I) in which R⁶ is other than hydrogen, the isomers can be separated by chromatographic methods (Scheme 2).

Compounds wherein one or more of R², R³, R⁴ and R⁵ is bromo can be prepared as described in Scheme 1. These compounds can be further reacted with aniline in the presence of trimethyl-aluminium to afford compounds where one or more of R², R³, R⁴ and R⁵ is aniline (see e.g. Scheme 3).

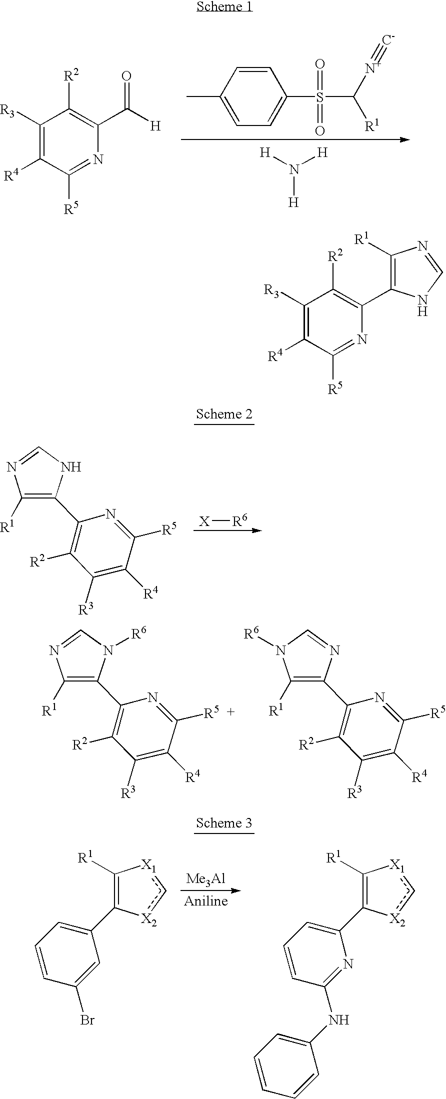

Further details for the preparation of compounds of formula (I) are found in the examples.

During the synthesis of the compounds of formula (I) labile functional groups in the intermediate compounds, e.g. hydroxy, carboxy and amino groups, may be protected. A comprehensive discussion of the ways in which various labile functional groups may be protected and methods for cleaving the resulting protected derivatives is given in for example Protective Groups in Organic Chemistry, T. W. Greene and P. G. M. Wuts, (Wiley-Interscience, New York, 2nd edition, 1991).

The compounds of formula (I) may be prepared singly or as compound libraries comprising at least 2, for example 5 to 1,000 compounds, and more preferably 10 to 100 compounds of formula (I). Libraries of compounds of formula (1) may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I) or pharmaceutically acceptable salts thereof.

According to a further aspect of the present invention there is provided a method of treating a disease mediated by the ALK5 receptor in mammals, comprising administering to a mammal in need of such treatment, an effective amount of a compound of formula (I) or a pharmaceutically acceptable salt thereof.

In another aspect of the invention there is provided the use of a compound of formula (I), or a pharmaceutically acceptable salt or solvate thereof, in therapy.

According to a further aspect of the present invention there is provided the use of a compound of formula (I) or a pharmaceutically acceptable salt thereof, in the manufacture of a medicament for the treatment of a disease mediated by the ALK5 receptor in mammals.

ALK5-mediated disease states, include, but are not limited to, chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal abrasion, any disease wherein fibrosis is a major component, including, but not limited to lung fibrosis, liver fibrosis and renal fibrosis, and restenosis.

By the term “treating” is meant either prophylactic or therapeutic therapy.

According to a further aspect of the present invention there is provided a method of inhibiting the TGF-β signaling pathway in mammals, for example, inhibiting the phosphorylation of smad2 or smad3 by the type I or activin-like kinase ALK5 receptor, which method comprises administering to a mammal in need of such treatment, an effective amount of a compound of formula (I) or a pharmaceutically acceptable salt thereof.

According to a further aspect of the present invention there is provided a method of inhibiting matrix formation in mammals by inhibiting the TGF-β signalling pathway, for example, inhibiting the phosphorylation of smad2 or smad3 by the type I or activin-like kinase ALK5 receptor, which method comprises administering to a mammal in need of such treatment, an effective amount of a compound of formula (I) or a pharmaceutically acceptable salt thereof.

The compounds of formula (I) and pharmaceutically acceptable salts thereof, may be administered in conventional dosage forms prepared by combining a compound of formula (1) with standard pharmaceutical carriers or diluents according to conventional procedures well known in the art. These procedures may involve mixing, granulating and compressing or dissolving the ingredients as appropriate to the desired preparation.

According to a further aspect of the present invention there is provided a pharmaceutical composition comprising a compound of formula (I), or a pharmaceutically acceptable salt thereof, and a pharmaceutically acceptable carrier or diluent.

The pharmaceutical compositions of the invention may be formulated for administration by any route, and include those in a form adapted for oral, topical or parenteral administration to mammals including humans.

The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognized by one of skill in the art that the optimal quantity and spacing of individual dosages of a formula (I) compound will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of the formula (I) compound given per day for a defined number of days, can be ascertained by those skilled in the art using conventional course of treatment determination tests.

No toxicological effects are indicated when a compound of formula (1) or a pharmaceutically acceptable derivative thereof is administered in the above-mentioned dosage range.

All publications, including, but not limited to, patents and patent applications cited in this specification, are herein incorporated by reference as if each individual publication were specifically and individually indicated to be incorporated by reference herein as though fully set forth.

The following examples are to be construed as merely illustrative and not a limitation on the scope of the invention in any way.

### EXAMPLE 1

### 2-[5-(4-Fluorophenyl)-1H-imidazol-4-yl]-pyridine

2M ammonia in methanol (9 ml, 18 mmol) was added to pyridine-2-carboxaldehyde (481 mg, 4.5 mmol) and stirred at room temperature for 2 h. 1-[1-Isocyano-1-(toluene-4-sulfonyl)-methyl]-4-fluorobenzene (1.89 g, 6 mmol) (prepared according to the method of J. Sisko et al; Tet. Letters, 1996, 37(45), 8113) and dry THF (8 ml) were added and stirring continued at room temperature for 48 h. The reaction mixture was diluted with ethyl acetate and washed with aqueous sodium carbonate then brine. The organic phase was dried (Na₂SO₄) and concentrated in vacuo. The residue was chromatographed on silica gel eluting with 2% methanol in dichloromethane to afford the title compound. ¹H NMR (250 MHz, CDCl₃) δ: 3.53 (3H, s), 7.03 (1H, m), 7.15 (2H, m), 7.36 (2H, m), 7.57 (2H, m), 7.60 (1H, s), 8.42 (1H, s).

The following compounds were prepared according to the method of Example 1 from the starting materials indicated:

### EXAMPLE 2

### 3-[5-(4-Fluorophenyl)-1H-imidazol-4-yl]-pyridine

From pyridine-3arboxaldehyde and 1-[1-isocyano-1-(toluene-4-sulfonyl)-methyl]-4-fluorobenzene. m/z (ES): 240 (M+H)⁺.

### EXAMPLE 3

### 2-(5-Benzo[1,3]dioxol-5-yl-1H-imidazol-4-yl)-pyridine

From pyridine-2-carboxaldehyde and 5-[1-isocyano-1-(toluene-4-sulfonyl)-methyl]-benzo[1,3]dioxole. m/z (ES): 266 (M+H)⁺.

### EXAMPLE 4

### 2-(5-Benzo[1,3]dioxol-5-yl-1H-imidazol-4-yl)-6-methyl-pyridine

From 6-methylpyridine-2-carboxaldehyde and 5-[1-isocyano-1-(toluene-4-sulfonyl)-methyl]-benzo[1,3]dioxole. m/z (ES): 280 (M+H)⁺.

### EXAMPLE 5

### 2-(5-Benzo[1,3]dioxol-5-yl-1H-imidazol-4-yl)-6-bromo-pyridine

From 6-bromo-pyridine-2-carboxaldehyde (prepared according to the method of Uenishi et al; Tet. Letters, 1994, 35(43), 7973) and 5-[1-isocyano-1-(toluene-4-sulfonyl)-methyl]-benzo[1,3]dioxole. m/z (ES): 318 (M+H)⁺.

### EXAMPLE 6

### 2-[5-(2,3-Dihydrobenzo[1,4]dioxin-6-yl)-1H-imidaz l-4-yl]-6-methyl-pyridine

From 6-bromo-pyridine-2-carboxaldehyde and 6-[1-isocyano-1-(toluenesulfonyl)-methyl]-2,3-dihydobenzo[1,4]dioxine. ¹H NMR (250 MHz, CDCl₃) δ: 2.53 (3H, s), 4.28 (4H, m), 6.89 (1H, d), 6.94 (1H, dd), 7.10 (1H, dd), 7.17 (1H, d), 7.36 (2H, m), 7.67 (1H, s); m/z (API⁺): 294 (MH)⁺.

### Example 7

### 2-[5-(4-Fluorophenyl)-3H-imidazol-4-yl]-quinoline

From quinoline-2-carboxaldehyde and 1-[1-isocyano-1-(toluene-4-sulfonyl)-methyl]-4-fluorobenzene. m/z (ES): 290 (MH⁺).

### EXAMPLE 8

### 2-[5-(2,3-Dihydro-benzo[1,4]dioxin-6-yl)-3-methyl-3H-imidazol-4-yl]-6-methyl-pyridinie

2-[5-(2,3-Dihydro-benzo[1,4]dioxin-6-yl)-1H-imidazol4-yl]-6-methyl-pyridine (250 mg, 0.85 mmol) and dimethyl formamide dimethyl acetal (0.3 ml) were added to toluene (15 ml) and heated at reflux for 48 h. On cooling, volatiles were removed in vacuo and the residue subjected to dry flash chromatography on silica gel eluting with 20% hexane in ethyl acetate.

¹H NMR (250 MHz, CDCl₃) δ: 2.62 (3H, s), 3.66 (3H, s), 4.22 (4H, m), 6.75 (1H, d), 6.92 (1H, dd), 7.00 (1H, d), 7.08 (2H, m), 7.53 (2H, m); m/z (API⁺): 308 (MH)⁺.

### EXAMPLE 9

### 2-[5-(2,3-Dihydro-benzo[1,4]dioxin-6-yl)-1-methyl-1H-imidazol-4-yl]-6-methyl-pyridine

2-[5-(2,3-Dihydro-benzo[1,4]dioxin-6-yl)-1H-imidazol-4-yl]-6-methyl-pyridine (200 mg, 0.68 mmol) was dissolved in dry THF under argon and cooled to 0° C. Sodium bis (trimethylsilyl) amide (0.75 ml, 1M in THF) was added dropwise and stirring continued at 0° C. for 15 min. The cooling bath was removed and stirring continued at room temperature for 1 h. The reaction mixture was diluted with water and extracted with dichloromethane. The organic phase was dried (Na₂SO₄) and concentrated in vacuo. The residue was subjected to dry flash chromatography on silica gel eluting with 2% methanol in dichloromethane to afford the title compound as a yellow oil (159 mg). ¹H NMR (250 MHz, CDCl₃) δ: 2.49 (3H, s), 3.50 (3H, s), 4.30 (4H, m), 6.89 (4H, m), 7.13 (1H, d), 7.37 (1H, t), 7.56 (1H, s); m/z (API⁺): 308 (MH)⁺.

### EXAMPLE 10

### [6-(5-Benzo[1,3]dioxol-5-yl-1H-imidazol-4-yl)-pyridin-2-yl]-phenylamine

To 2-(5-Benzo[1,3]dioxol-5-yl-1H-imidazol-4-yl)-6-bromo-pyridine (52 mg, 0.15 mmol) under argon was added aniline (55 ul, 0.6 mmol) followed by toluene (700 ul) then trimethylaluminum (0.3 ml, 2M in toluene, 0.6 mmol). The resultant mixture was heated overnight in a sealed tube at 90° C. On cooling, the reaction mixture was poured into 10% aqueous NaOH and extracted with ethyl acetate. The organic phase was dried (MgSO₄) and concentrated in vacua. The residue was subjected to dry flash chromatography on silica gel eluting with 4% propan-2-ol in dichloromethane to afford the title compound as a yellow oil. m/z (ES): 257 (M+H)⁺.

### EXAMPLE 11

### {6-[5-(4-Fluorophenyl)-1H-imidazol-4-yl]-pyridin-2-yl}-phenylamine Pyrimidine

Prepared according to the procedure for Example 10 from 2-[5-(4-fluorophenyl)-1H-inidazol-4-yl]-6-bromo-pyridine and aniline. m/z (ES): 331 (M+H)⁺.

Biological Data

The biological activity of the compounds may be assessed using the following assays:

Method for Evaluating ALK5 Kinase Phosphorylation of smad3

Basic Flash-Plates (NEN Life Sciences) were coated by pipetting 100 micro liter of 0.1 molar sodium bicarbonate (pH 7.6), containing 150 nanograms of the fusion protein glutathion-S-transferase-smad3/100 micro liter of coating buffer. Plates were covered and incubated at room temperature for 10-24 hours. Then the plates were washed 2 times with 200 micro liter of coating buffer (0.1 molar sodium bicarbonate) and allowed to air dry for 2-4 hours.

For the phosphorylation reaction each well received 90 microliter containing 50 millimolar HEPES buffer (pH 7.4); 5 millimolar MgCl₂; 1 millimolar CaCl₂; 1 millimolar dithiothreitol; 100 micromolar guanosine triphosphate; 0.5 micro Ci/well gamma³³P-adenosine triphosphate (NEN Life Sciences) and 400 nanograms of a fusion protein of glutathion S-transferase at the N-terminal end of the kinase domain of ALK5 (GST-ALK5). Background counts were measured by not adding any GST-ALK5. Inhibitors of ALK5 were evaluated by determining the activity of the enzyme in the presence of various compounds. Plates were incubated for 3 hours at 30° C. After incubation the assay buffer was removed by aspiration and the wells were washed 3 times with 200 microliter cold 10 millimolar sodium pyrophosphate in phosphate buffered saline. The last wash was aspirated and blotted plate dry. Plate was then counted on a Packard TopCount.

Inhibition of Matrix Markers: Western Blot Protocol

Data confirming activity in the enzyme assay was obtained as follows.

Cells were grown to near confluence in flasks, starved overnight and treated with TGF-beta and compounds. Cells were washed at 24 or 48 hours after treatment with ice cold phosphate buffered saline, then 500 microliter of 2× loading buffer was added to plate and cells were scraped and collected in microcentrifuge tube. (2× loading buffer: 100 mM TrisCl, pH 6.8, 4% sodium dodecyl sulfate, 0.2% bromophenol blue, 20% glycerol, 5% beta-mercapto-ethanol). Cells were lysed in tube and vortexed. Sample was boiled for 10 minutes. 20 microliters of sample was loaded on 7.5% polyacrylamide gel (BioRad) and electrophoresed.

Size fractionated proteins in gel were transferred to nitrocellulose membrane by semidry blotting. Membrane was blocked overnight with 5% powdered milk in phosphate buffer saline (PBS) and 0.05% Tween-20 at 4 degrees C. After 3 washes with PBS/Tween membranes were incubated with primary antibody for 4 hours at room temperature. After three washes with PBS/Tween membrane was incubated with secondary antibody for 1 hour at room temperature. Finally, a signal was visualized with ECL detection kit from Amersham.

The compounds generally show ALK5 receptor modulator activity having IC₅₀ values in the range of 0.0001 to 10 μm.

1. A compound of formula (I), or a pharmaceutically acceptable salt thereof: 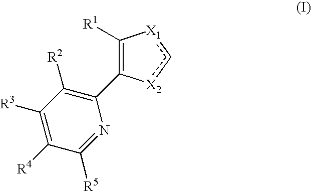
wherein R¹ is phenyl or naphthyl optionally substituted with one or more substituents selected from halo, C₁₋₆alkoxy, C₁₋₆alkylthio, C₁₋₆alkyl, C₁₋₆haloalkyl, —O—(CH₂)ₘ—Ph, —S—(CH₂)ₘ—Ph, cyano, phenyl, and CO₂R, wherein R is hydrogen or C₁₋₆alkyl and m is 0-3; or phenyl fused with a 5- to 7-membered aromatic or non-aromatic ring wherein said ring contains up to three heteroatoms, independently selected from N, O and S;
R², R³, R⁴ and R⁵ independently represent hydrogen, C₁₋₆alkyl, C₁₋₆alkoxy, C₁₋₆haloalkyl, halo, NH₂, NH—C₁₋₆alkyl or NH(CH₂)ₙ—Ph wherein n is 0-3; or an adjacent pair of R², R³, R⁴ and R⁵ form a fused 6-membered aromatic ring optionally containing up to 2 nitrogen atoms, said ring being optionally substituted by one or more substituents independently selected from C₁₋₄alkyl, C₁₋₄alkoxy, C₁₋₆haloalkyl, halo, NH₂, NH—C₁₋₆allkyl or NH(CH₂)ₙ—Ph wherein n is 0-3, and the remainder of R², R³, R⁴ and R₅ represent hydrogen, C₁₋₆alkyl, C₁₋₆alkoxy, C₁₋₆haloalkyl, halo, NH₂, NH—C₁₋₆alkyl or NH(CH₂)ₙ—Ph wherein n is 0-3; and
one of X₁ and X₂ is N and the other is NR₆, wherein R⁶ is hydrogen or C₁₋₆alkyl.

2. A compound of according to claim 1 wherein R¹ is phenyl optionally substituted by halo, or R¹ is phenyl fused with a 5- to 7-membered aromatic or non-aromatic ring wherein said ring optionally contains up to three heteroatoms, independently selected from N, O and S.

3. A compound according to claim 1 or 2 wherein one of R², R³, R⁴ and R⁵ is other than hydrogen.

4. A compound according to any one of the preceding claims wherein R⁵ is methyl or halo.

5. A compound according to any one of the preceding claims wherein R⁶ is hydrogen.

6. A compound according of formula (I) as defined in any one of Examples 1 to 11 or a pharmaceutically acceptable salt thereof.

7. A pharmaceutical composition comprising a compound according to any one of the preceding claims, or a pharmaceutically acceptable salt thereof, and a pharmaceutically acceptable carrier or diluent.

8. A method for treating a disease mediated by the ALK5 receptor in mammals, comprising administering to a mammal in need of such treatment, a therapeutically effective amount of a compound according to any one of claims 1 to 6, or a pharmaceutically acceptable salt thereof.

9. A method for treating a disease selected from chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component including lung fibrosis, liver fibrosis and renal fibrosis, and restenosis, comprising administering to a mammal in need of such treatment, a therapeutically effective amount of a compound according to any one of claims 1 to 6, or a pharmaceutically acceptable salt thereof.

10. A method for inhibiting matrix formation in mammals, comprising administering to a mammal, a therapeutically effective amount of a compound according to any one of claims 1 to 6, or a pharmaceutically acceptable salt thereof.

11. The use of a compound of formula (I) as claimed in any one of claims 1 to 6, or a pharmaceutically acceptable salt or solvate thereof, in therapy.

12. The use of a compound of formula (I) as claimed in any one of claims 1 to 6, or a pharmaceutically acceptable salt thereof, in the manufacture of a medicament for the treatment of a disease mediated by the ALK5 receptor in mammals.
